(19) United States
(12) Patent Application Publication
(10) Pub. No.: US 2019/0033787 A1
(43) Pub. Date: Jan. 31, 2019

(54) DEVICE AND METHOD FOR EMITTING LIGHT AWAY FROM A USER

(71) Applicants: Evan Swanagin, Birmingham, AL (US); Stephen Swanagin, Birmingham, AL (US)

(72) Inventors: Evan Swanagin, Birmingham, AL (US); Stephen Swanagin, Birmingham, AL (US)

(21) Appl. No.: 16/045,575

(22) Filed: Jul. 25, 2018

Related U.S. Application Data
(60) Provisional application No. 62/536,981, filed on Jul. 25, 2017.

(51) Int. Cl. G04B 19/30; F21L 4/02; F21V 21/088; F21V 21/14; A61B 5/00

(52) U.S. Cl. CPC G04B 19/30; F21L 4/02; A61B 5/6824; F21V 21/145; A61B 5/681; F21V 21/0885

(57) ABSTRACT

A device for emitting light away from a user is disclosed herein. The device includes an elongate body having a main segment, a light source, an energy storage, a clasping mechanism, and a user interface. The user interface includes a depressible element configured to switch the light source on and off. The depressible element is positioned on the top surface of the main segment.

### CROSS-REFERENCE TO RELATED APPLICATION(S)

The present application is related to and claims priority to U.S. Provisional Patent Application No. 62/536,981 filed Jul. 25, 2017, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of illumination devices of existing art and more specifically relates to wrist-worn lighting device.

### RELATED ART

There are many instances where individuals find themselves performing tasks or activities in the dark. Without proper lighting, these tasks can be dangerous. Running or biking at night, working on job sites after dark, camping, and other activities can be difficult to do without proper lighting. However, in order to obtain light, it typically requires the user to hold a flashlight or their cell phone. This does not allow the individual to utilize both hands to complete the task at hand. A suitable solution is desired.

U.S. Pub. No. 2005/0254229 to Ali Hamade and Amanda Pohan relates to an I-light. The described I-light includes a powerful long-lasting flashlight, watch, compass, and GPS tracking device all combined into one small and sleek fusion of rubber, glass and titanium that is worn on the wrist like a watch. A flashlight cannot accomplish all of these things, and even if it could, it would still be a burden because it would be yet another object that you have to drag around with you. A flashlight that is worn on the head with a strap is not at all attractive or practical and does not include the other features that I-Light has. The I-Light frees you from worrying about losing your flashlight or lugging it around. The I-Light is an exceptional and unique invention that has never been attempted before. The I-Light is unique, perfect, and practical, and we guarantee that it is truly one of a kind.

### SUMMARY OF THE INVENTION

In view of the foregoing disadvantages inherent in the known illumination device art, the present disclosure provides a novel device and method for emitting light away from a user. The general purpose of the present disclosure, which will be described subsequently in greater detail, is to provide a wearable LED flashlight that is worn around the wrist via a silicone band.

A device for emitting light away from a user is disclosed herein. The device may include an elongate body having a main segment, a light source, an energy storage, a clasping mechanism, and a user interface. The user interface may include a depressible element configured to switch the light source on and off. The depressible element may be additionally positioned on the top surface of the main segment.

According to another embodiment, a method of emitting light away from a user is also disclosed herein. The method of emitting light away from a user may include providing a device for emitting light, placing the device around a wrist of the user, turning on the light source via pressing the depressible element a first time, increasing a light intensity of the light source via pressing the depressible element a second time, and actuating a flashing sequence of light via pressing the depressible element a third time.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a device and method for emitting light away from a user, constructed and operative according to the teachings of the present disclosure.

FIG. 1 is a perspective view of the device for emitting light during an ‘in-use’ condition, according to an embodiment of the disclosure.

FIG. 2 is an alternate perspective view of the device for emitting light of FIG. 1, according to an embodiment of the present disclosure.

FIG. 3 is a front view of the device for emitting light of FIG. 1, according to an embodiment of the present disclosure.

FIG. 4 is a back view of the device for emitting light of FIG. 1, according to an embodiment of the present disclosure.

FIG. 5 is a flow diagram illustrating a method of emitting light away from a user, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a illumination device and more particularly to a device and method for emitting light away from a user as used to provide a wearable LED flashlight that is worn around the wrist via a silicone band.

Generally, a wearable LED flashlight that is worn around the wrist via a silicone band. It offers a light that is USB-rechargeable and waterproof and has multiple operation modes. This appeals to runners, bikers, campers, hikers, plumbers, hunters, and any other user who desires hands-free light in dimly lit locations. It comprises a sleek and narrow profile to avoid being bulky and cumbersome on the wrist. The present invention allows users to safely perform activities or tasks in the dark by providing ample light.

The device may include a light mounted to a wristband to allow users to perform activities or tasks in the dark without the need to hold a flashlight. The invention is comprised of a durable silicone band that encloses a fixture that contains an LED strip angling about 45 degrees away from the body. The USB port will be protected by a silicone tab. The on/off button will be build under the silicone to ensure that it is waterproof. The band will be assembled with a classic watch-like clasp/closure. The device is powered by a lithium battery that can be recharged via the USB port. The on/off button has two operational modes. These modes will include a high-beam light on the first click and strobe light on the second click, and a third click turns the device off. The device should have a life span of three to nine hours. The exact specifications may vary.

In a preferred embodiment, the device may include a wearable flashlight for a wrist of a user, the wearable flashlight comprising a wristband configured to couple to the wrist, the wrist band defining a forward direction as being toward a hand of the user and away from a forearm of the user; a light fixed to the wristband and configured to emit light in the forward direction; an energy storage fixed to the wristband and configured to power the light; and a user interface including a depressible element configured to switch the light on and off.

Upon reading this specification, it should be appreciated that, under appropriate circumstances, considering such issues as user preferences, design preference, structural requirements, marketing preferences, cost, available materials, technological advances, etc., other wearable flashlight arrangements such as, for example, a wristband, etc., may be sufficient.

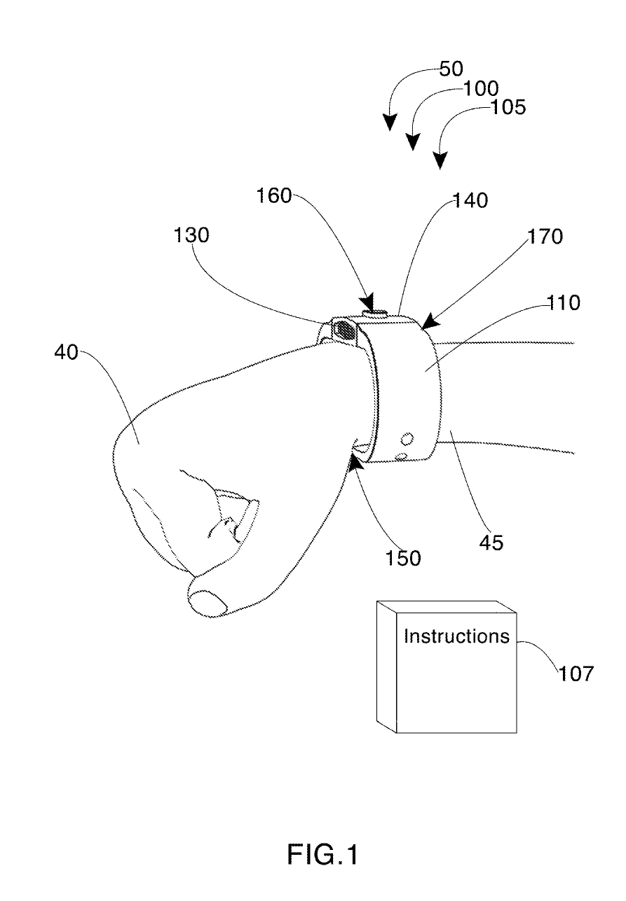

Referring now more specifically to the drawings by numerals of reference, there is shown in FIGS. 1-4, various views of a device 100 for emitting light away from a user 40.

FIG. 1 shows a device 100 for emitting light away from a user 40 during an ‘in-use’ condition 50, according to an embodiment of the present disclosure. Here, the device 100 may be beneficial for use by a user 40 to provide a wearable LED flashlight that is worn around the wrist via a silicone band. As illustrated, the device 100 may include an elongate body 110, a light source 130, an energy storage 140, a clasping mechanism 150, a user interface 160, and a charging port 170. The device 100 may be configured to be worn about a wrist 45 of the user 40.

According to one embodiment, the device 100 for emitting light away from a user 40 may be arranged as a kit 105. In particular, the device 100 may further include a set of instructions 107. The instructions 107 may detail functional relationships in relation to the structure of the device 100 such that the device 100 can be used, maintained, or the like, in a preferred manner.

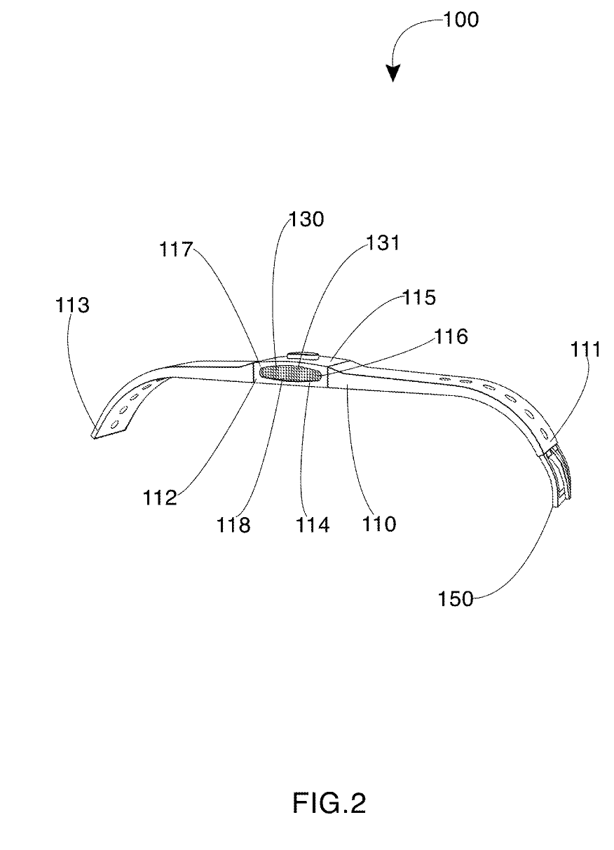

FIG. 2 shows the device 100 for emitting light away from a user 40 of FIG. 1, according to an embodiment of the present disclosure. As above, the device 100 may include an elongate body 110 having a first end segment 111, a main segment 112, and a second end segment 113. The main segment 112 may have a front side 114, a top surface 115, an inside 116, and an outside 117. The elongate body 110 may further include a light reflecting screen 118 located on the front side 114 of the main segment 112. The first end segment 111 may include a clasping mechanism 150 that is configured to fasten the first end segment 111 and the second end segment 113 together. Further still, the device 100 may include a light source 130 located inside the main segment 112 that is configured to emit light.

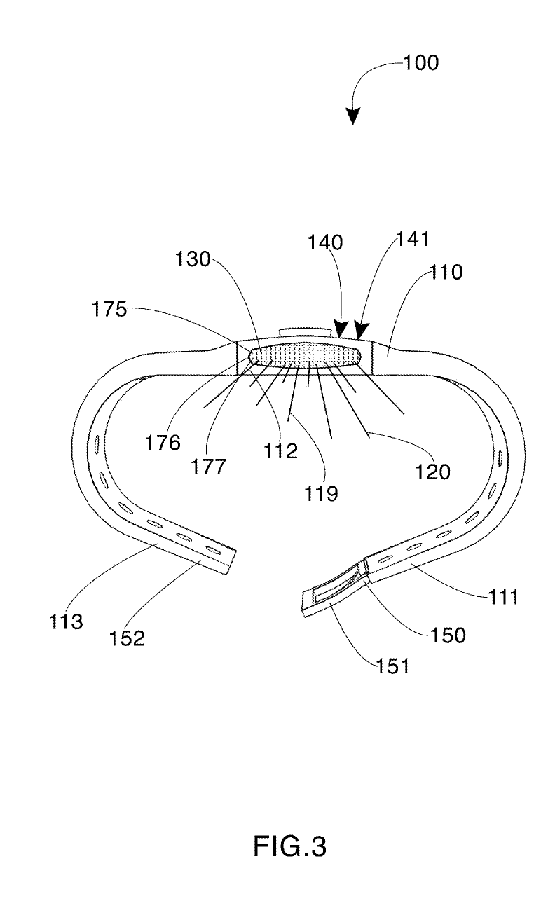

FIG. 3 is a front view of the device 100 for emitting light away from a user 40 of FIG. 1, according to an embodiment of the present disclosure. Here again, the device 100 may include an elongate body 110 having a first end segment 111, a main segment 112, and a second end segment 113. The device 100 may further include a light source 130 located in the main segment 112. The light source 130 may be configured to emit light such that an emitted light 119 is reflected at a predetermined angle 120 away from the user 40 (not shown). In a preferred embodiment, the predetermined angle 120 may be approximately 45 degrees. The device 100 may also include a clasping mechanism 150 configured to fasten the first end segment 111 and the second end segment 113 together. The clasping mechanism may include a tang buckle 151 and a plurality of apertures 152. In addition, the device may have an energy storage 140 that includes a lithium-ion battery 141. Further still, the device 100 may include a plurality of modes of operation 175. The plurality of modes of operation 175 may include at least two light intensity levels 176 and a flashing mode 177.

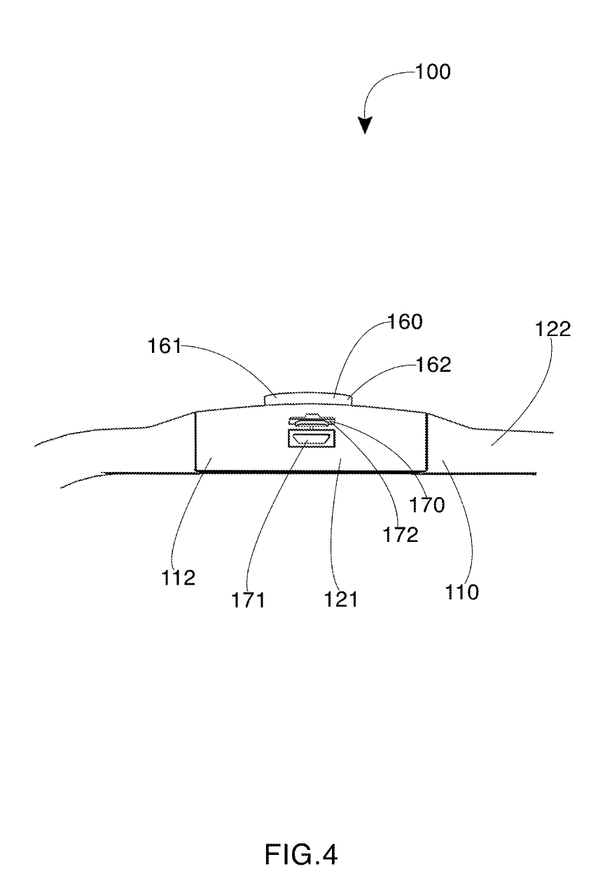

FIG. 4 is a back view of the device 100 for emitting light away from a user 40 of FIG. 1, according to an embodiment of the present disclosure. As above, the device 100 may include an elongate body 110 having a main segment 112. The elongate body 110 may be arranged as a wristband 122. The device 100 may also include a charging port 170 positioned on a back side 121 of the main segment 112. The charging port 170 may include a USB port 171 along with a cover configured to protect the USB port 171. Finally, the device 100 may have a user interface 160 including a depressible element 161 that is configured to switch the light source on and off. The depressible element 161 may be positioned on the top surface of the main segment 112. The depressible element 161 may include a push button 162 configured to turn the device 100 on and off.

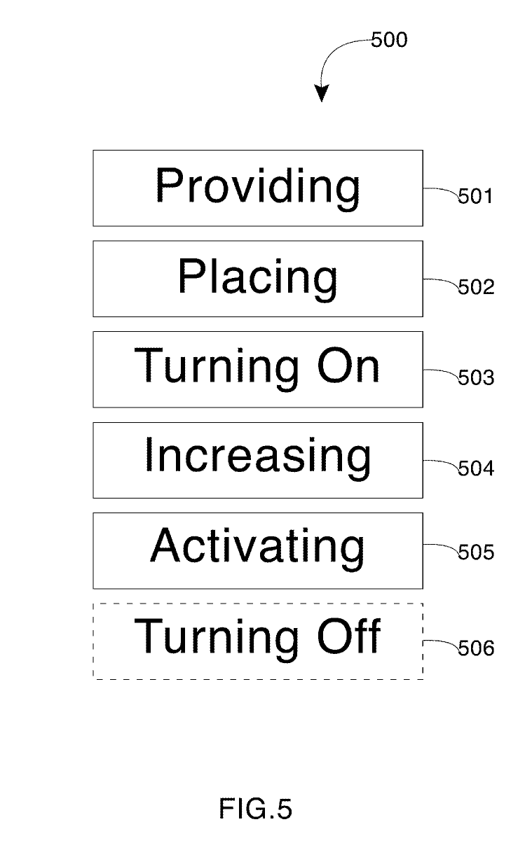

FIG. 5 is a flow diagram illustrating a method 500 of emitting light away from a user 40, according to an embodiment of the present disclosure. In particular, the method 500 of emitting light away from a user 40 may include one or more components or features of the device 100 as described above. As illustrated, the method 500 of emitting light away from a user 40 may include the steps of: step one 501, providing a device for emitting light, said device including an elongate body, the elongate body having a first end segment, a main segment, and a second end segment, the main segment having a front side, a top surface, an inside, and an outside, the elongate body including a light reflecting screen located on the front side of the main segment, said light reflecting screen configured to reflected an emitted light at a predetermined angle away from the user, a light source, the light source located inside the main segment and configured to emit light, such that the emitted light is reflected at the predetermined angle away from the user, an energy storage, the energy storage located inside the main segment and configured to provide power to the light source, a clasping mechanism, the clasping mechanism configured to fasten the first end segment and the second end segment together, and a user interface, the user interface including a depressible element configured to switch the light source on and off, the depressible element positioned on the top surface of the main segment; step two 502, placing the device around a wrist of the user; step three 503, turning on the light source via pressing the depressible element a first time; step four 504, increasing a light intensity of the light source via pressing the depressible element a second time; and step five 505, actuating a flashing sequence of light via pressing the depressible element a third time.

It should be noted that step six 506, turning the device off via pressing the depressible element a fourth time, is an optional step and may not be implemented in all cases. Optional steps of method of use 500 are illustrated using dotted lines in FIG. 5 so as to distinguish them from the other steps of method of use 500. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for a device for emitting light away from a user, are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A device for emitting light away from a user, the device comprising:
an elongate body, the elongate body having a first end segment, a main segment, and a second end segment, the main segment having a front side, a top surface, an inside, and an outside, the elongate body including a light reflecting screen located on the front side of the main segment, said light reflecting screen configured to reflect an emitted light at a predetermined angle away from the user;
a light source, the light source located inside the main segment and configured to emit light, such that the emitted light is reflected at the predetermined angle away from the user;
an energy storage, the energy storage located inside the main segment and configured to provide power to the light source;
a clasping mechanism, the clasping mechanism configured to fasten the first end segment and the second end segment together; and
a user interface, the user interface including a depressible element configured to switch the light source on and off, the depressible element positioned on the top surface of the main segment.

2. The device of claim 1, wherein the energy storage includes a lithium-ion battery.

3. The device of claim 1, wherein the elongate body is arranged as a wristband, and the first end segment and the second end segment are positioned opposite each other relative to the main segment, and together, the first end segment, the second end segment, and the main segment are configured to be worn about a wrist of the user.

4. The device of claim 2, wherein the elongate body is made of a silicone.

5. The device of claim 4, wherein the depressible element is covered by the silicone.

6. The device of claim 1, wherein the depressible element includes a push button configured to turn the device on and off.

7. The device of claim 1, wherein the device includes a plurality of modes of operation.

8. The device of claim 7, wherein the plurality of modes of operation include at least two light intensity levels and a flashing mode.

9. The device of claim 1, wherein the clasping mechanism includes a tang buckle and a plurality of apertures.

10. The device of claim 8, wherein the plurality of apertures is positioned on the second end segment.

11. The device of claim 9, wherein the tang buckle is positioned on the first end segment.

12. The device of claim 1, wherein the device is water-proof.

13. The device of claim 1, further comprising a charging port, said charging port positioned on a back side of the main segment.

14. The device of claim 13, wherein the charging port includes a USB port and a cover configured to protect the USB port.

15. The device of claim 1, wherein the light source includes a light emitting diode (LED) strip.

16. The device of claim 1, wherein the predetermined angle is approximately 45 degrees as measured from top surface in a forward direction.

17. A device for emitting light away from a user, the device comprising:
an elongate body, the elongate body having a first end segment, a main segment, and a second end segment, the main segment having a front side, a top surface, an inside, and an outside, the elongate body including a light reflecting screen located on the front side of the main segment, said light reflecting screen configured to reflect an emitted light at a predetermined angle away from the user;
a light source, the light source located inside the main segment and configured to emit light, such that the emitted light is reflected at the predetermined angle away from the user;
an energy storage, the energy storage located inside the main segment and configured to provide power to the light source;
a clasping mechanism, the clasping mechanism configured to fasten the first end segment and the second end segment together;
a user interface, the user interface including a depressible element configured to switch the light source on and off, the depressible element positioned on the top surface of the main segment; and
a charging port, said charging port positioned on a back side of the main segment; and
wherein the energy storage includes a lithium-ion battery;
wherein the elongate body is arranged as a wristband, and the first end segment and the second end segment are positioned opposite each other relative to the main segment, and together, the first end segment, the second end segment, and the main segment are configured to be worn about a wrist of the user;
wherein the elongate body is made of silicone;
wherein the depressible element is covered by the silicone;
wherein the depressible element includes a push button configured to turn the device on and off;
wherein the device includes a plurality of modes of operation;
wherein the plurality of modes of operation include at least two light intensity levels and a flashing mode;
wherein the clasping mechanism includes a tang buckle and a plurality of apertures;
wherein the plurality of apertures is positioned on the second end segment;
wherein the tang buckle is positioned on the first end segment;
wherein the device is water-proof;
wherein the charging port includes a USB port and a cover configured to protect the USB port;
wherein the light source includes a light emitting diode (LED) strip; and
wherein the predetermined angle is approximately 45 degrees as measured from top surface in a forward direction.

18. The device of claim 17, further comprising set of instructions; and
wherein the device for emitting light is arranged as a kit.

19. A method of emitting light away from a user, the method comprising the steps of:
providing a device for emitting light, said device including
an elongate body, the elongate body having a first end segment, a main segment, and a second end segment, the main segment having a front side, a top surface, an inside, and an outside, the elongate body including a light reflecting screen located on the front side of the main segment, said light reflecting screen configured to reflect an emitted light at a predetermined angle away from the user,
a light source, the light source located inside the main segment and configured to emit light, such that the emitted light is reflected at the predetermined angle away from the user,
an energy storage, the energy storage located inside the main segment and configured to provide power to the light source,
a clasping mechanism, the clasping mechanism configured to fasten the first end segment and the second end segment together, and
a user interface, the user interface including a depressible element configured to switch the light source on and off, the depressible element positioned on the top surface of the main segment;
placing the device around a wrist of the user;
turning on the light source via pressing the depressible element a first time;
increasing a light intensity of the light source via pressing the depressible element a second time; and
actuating a flashing sequence of light via pressing the depressible element a third time.

20. The method of claim 19, further comprising the step of turning the device off via pressing the depressible element a fourth time.
